(19) United States
(12) Patent Application Publication
(10) Pub. No.: US 2005/0130118 A1
(43) Pub. Date: Jun. 16, 2005

(54) Methods for identifying inhibitors of chlorophyll synthase

(76) Inventors: Abubakr Aslamkhan, Durham, NC (US); Lining Guo, Chapel Hill, NC (US); Rao Mulpuri, Apex, NC (US); Neil Hoffman, Chapel Hill, NC (US); Susanne Kjemtrup, Chapel Hill, NC (US); Jocelyn Kearney, Cary, NC (US); Cory A. Christensen, Cary, NC (US); Keith Davis, Durham, NC (US); Adel Zayed, Durham, NC (US); Robert Ascenzi, Cary, NC (US); Douglas Boyes, Chapel Hill, NC (US)

Correspondence Address: Icoria, Inc., 108 T.W. ALEXANDER DRIVE, P O BOX 14528, RTP, NC, 27709-4528, US

(21) Appl. No.: 10/737,014

(22) Filed: Dec. 16, 2003

(51) Int. Cl. C12Q 1/00

(52) U.S. Cl. 435/4

(57) ABSTRACT

The present inventors have discovered that antisense suppression of a chlorophyll synthase (CS) gene results in plants exhibiting one or more of chlorosis, reduced growth, and altered development. Thus, the present inventors have discovered that the protein encoded by the CS gene is essential for normal plant growth and development, and is useful as a target for the identification of compounds as antibiotics and herbicides, especially herbicides. The present invention is directed to methods for identifying inhibitors of a CS enzyme by incubating a CS polypeptide with a chlorophyllide and a phopholipid substrate in the presence and absence of a test compound under conditions suitable for the CS enzyme activity, adding a solution to the incubation reactions comprising a water immiscible organic solvent, a water-soluble alcohol, and a water-soluble dye that absorbs in the range of one or both the excitation and emission wavelength ranges of the chlorophyllide substrate, and measuring the fluorescence of the incubation reactions at from about 650 to 750 nm, using from about 425 to 445 nm as excitation wavelength, wherein a decrease in the fluorescence in the presence of the test compound indicates that the compound is a CS inhibitor.

### FIELD OF THE INVENTION

The invention relates to methods for the identification of inhibitors of chlorophyll synthase, particularly inhibitors useful as herbicides. The methods of the present invention are amenable for use in high throughput formats.

### BACKGROUND OF THE INVENTION

Chlorophyll synthase (CS) is a membrane bound protein localized in the thylakoid of higher plants (Lindsten et al. (1993) 88 Physiologia Plantarum. 29-36). The enzyme catalyzes the esterification of propionate moiety at position 17 of the chlorin ring in chlorophyllide (chelide) with either geranylgeranyl (in etiolated seedlings) or phytyl diphosphate (in green plants) resulting in the formation of chlorophyll a (see FIG. 1) (Schmid et al. (2002) 383 Biol. Chem. 1769-78). Chlorophyll synthase, like protochlorophyllide oxidoreductase, has an absolute requirement for the presence of an R conformation at position 13² of the isocyclic ring of chlorophyllide (FIG. 1) (Helfrich et al. (1994) 219 Eur. J. Biochem. 267-75).

Enolization of the proton at the 13² position of chlorophyllide occurs in the presence of polar organic solvents that can act as Lewis base (e.g. ethanol, acetone, diethyl ether, etc.) resulting in epimerization of the 13² carbon to an S conformation, and hence inactivation of the substrate (Helaja, J. (2000) Structural Analysis of Natural Chlorin Derivatives Utilizing NMR Spectroscopy and Molecular Modelling, University of Helsinki, Finland). As the solvents listed above are utilized in existing procedures for chlorophyllide purification, any given lot of purified substrate contains a certain proportion of non-reactive chlorophyllide.

The activity and substrate specificity of chlorophyll synthase from higher plants were studied by heterologous expression in E. coli using CS from Avena sativa (oat) (Schmid et al. (2001) 382 Biol. Chem. 903-11) and CS from Arabidopsis thaliana (Oster, U. & Rudiger, W. (1997) 110 Bot. Acta. 420-3). The Arabidopsis enzyme preferred use of geranylgeranyl diphosphate as a substrate to use of phytyl diphosphate. Kinetic analysis of the oat protein revealed that the CS uses a “ping pong” mechanism in esterification of chlorophyllide, with phytyl diphosphate binding to the active site as the first substrate (Schmid et al. (2002), supra).

The present invention discloses efficient methods that are amenable to a high throughput format, for the identification of compounds that are inhibitors of CS and that function as antibiotics and herbicides, especially herbicides.

### SUMMARY OF THE INVENTION

The present inventors have discovered that antisense suppression of a chlorophyll synthase (CS) gene results in plants exhibiting one or more of chlorosis, reduced growth, and altered development. Thus, the present inventors have discovered that the protein encoded by the CS gene is essential for normal plant growth and development, and is useful as a target for the identification of compounds as antibiotics and herbicides, especially herbicides. The present invention is directed to methods for identifying inhibitors of a CS enzyme by incubating a CS polypeptide with a chlorophyllide and a phopholipid substrate in the presence and absence of a test compound under conditions suitable for the CS enzyme activity, adding a solution to the incubation reactions comprising a water immiscible organic solvent, a water-soluble alcohol, and a water-soluble dye that absorbs in the range of one or both the excitation and emission wavelength ranges of the chlorophyllide substrate, and measuring the fluorescence of the incubation reactions at from about 650 to 750 nm, using from about 425 to 445 nm as excitation wavelength, wherein a decrease in the fluorescence in the presence of the test compound indicates that the compound is a CS inhibitor.

In the methods of the present invention, addition of a solution comprising a water immiscible organic solvent and a dye is used to advantage to selectively suppress fluorescence of the CS chlorophyllide substrate over the CS chlorophyll product. The selective suppression is achieved by partitioning the chlorophyllide substrate and the dye into an aqueous phase and the product into a separate organic phase, such that only the substrate is subjected to the interfering properties of the dye. In this manner, the invention provides an efficient method for identifying CS inhibitory compounds that does not require manipulation of the incubation reactions to physically separate the product-containing organic phase from the substrate-containing aqueous phase. Thus, the methods of the present invention enable the concurrent testing of multiple compounds using a high throughput format such as with 96- or 384-well plates. The CS polypeptides of the invention include plant CS polypeptides and in particular, Arabidopsis thaliana CS polypeptide.

### BRIEF DESCRIPTION OF THE FIGURES

FIG. 1. Diagram of the reaction catalyzed by chlorophyll synthase (CS). The enzyme catalyzes the esterification of chlorophyllide using either phytyl or geranylgeranyl diphosphate (shown). The latter substrate is utilized in etiolated seedlings, resulting in the formation of chlorophyll with a geranylgeranyl tail. Three double bonds in the product are reduced by geranylgeranyl reductase converting the product to phytyl chlorophyll, also known as chlorophyll a.

FIGS. 2A-2B. Depiction of a method in which a water-soluble dye, Malachite Green, is used to selectively quench the fluorescence of s substrate chlorophyllide (chelide) in an assay for CS enzyme activity. FIG. 2A shows a graph of fluorescence intensity (left y-axis) and absorbance (right y-axis) versus wavelength range 400-800 nm (x-axis) for the chelide substrate and the Malachite Green dye. The fluorescence excitation wavelength and emission range for the chelide substrate are each labeled with arrows on the graph and the absorbance spectrum of the Malachite Green dye from 400-800 nm is also depicted. As shown in FIG. 2A, the spectral properties of the Malachite Green dye enable it to act as an inner filter that minimizes fluorescence of the chelide substrate by absorbing both excitation and emission photons. FIG. 2B depicts the two-phase method employed in the CS activity assay of the invention to selectively measure fluorescence due to product (chlorophyll). The chelide substrate and dye remain in the aqueous phase while the product chlorophyll partitions into the organic phase. As a result, fluorescence measured at 665 nm using a 440 nm excitation wavelength is selectively inhibited for the chelide substrate but not for the product chlorophyll.

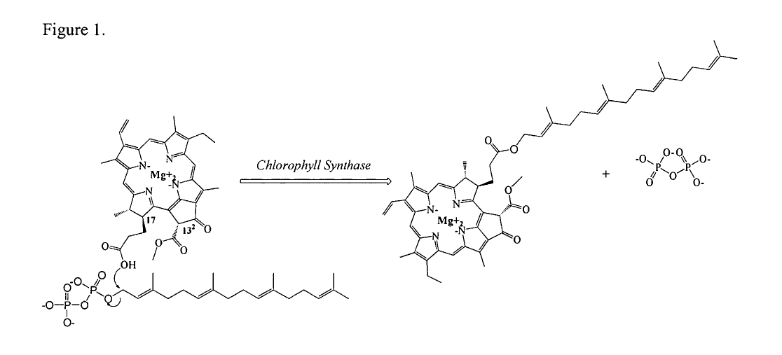

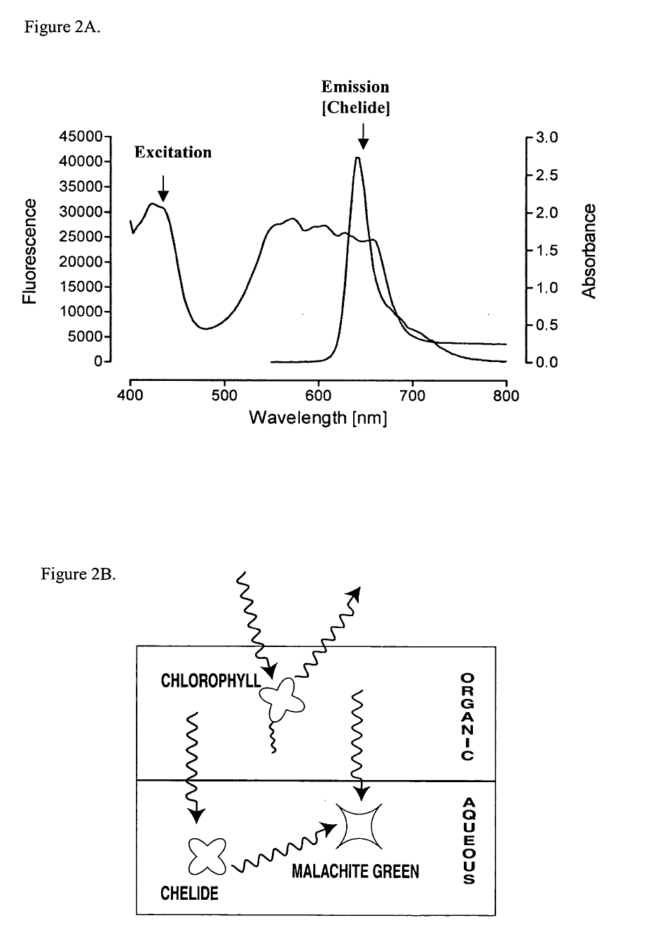

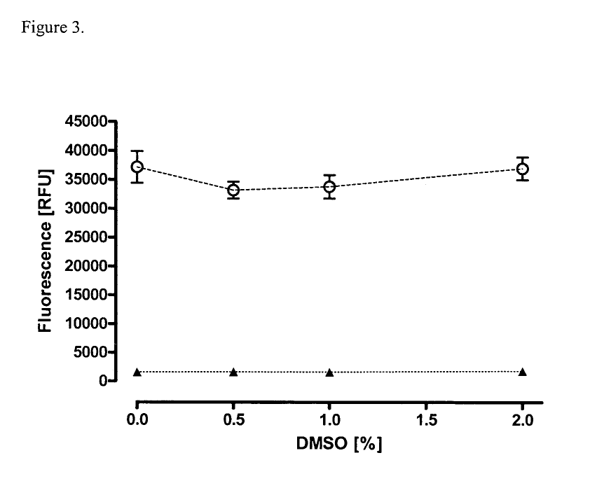

### DETAILED DESCRIPTION OF THE INVENTION

Definitions

A term not otherwise defined is intended to have its ordinary meaning.

The term “binding” refers to a non-covalent interaction that holds two molecules together. For example, two such molecules could be an enzyme and an inhibitor of that enzyme. Noncovalent interactions include hydrogen bonding, ionic interactions among charged groups, van der Waals interactions and hydrophobic interactions among nonpolar groups. One or more of these interactions mediates the binding of two molecules to each other.

As used herein, the term “cDNA” means complementary deoxyribonucleic acid.

As used herein, the term “CS” refers to a polypeptide that catalyzes the esterification of propionate moiety at position 17 of the chlorin ring in chlorophyllide (chelide) with a phospholipid substrate such as geranylgeranyl diphosphate or phytyl diphosphate, resulting in the formation of chlorophyll a. The CS polypeptide has an absolute requirement for the presence of an R conformation at position 13² of the isocyclic ring of the chlorophyllide. The CS polypeptides of the invention include, but are not limited to, Arabidopsis thaliana CS (SEQ ID NO:1); Oryza sativa CS (SEQ ID NO:2); Avena sativa CS (SEQ ID NO:3); and Arabidopsis thaliana CS fusion protein (SEQ ID NO:4).

As used herein, the term “GUS” means β-glucouronidase.

The term “herbicide”, as used herein, refers to a compound useful for killing or suppressing the growth of at least one plant, plant cell, plant tissue or seed.

The term “inhibitor,” as used herein, refers to a chemical substance that eliminates or substantially reduces CS enzymatic activity, wherein “substantially” means a reduction at least as great as the standard deviation for a measurement, preferably a reduction by 50%, more preferably a reduction of at least one magnitude, i.e. to 10%.

As used herein, the term “LB” means Luria-Bertani media.

As used herein, the term “Ni-NTA” refers to nickel sepharose.

As used herein, the term “PCR” means polymerase chain reaction.

“Plant” refers to whole plants, plant organs and tissues (e.g., stems, roots, ovules, stamens, leaves, embryos, meristematic regions, callus tissue, gametophytes, sporophytes, pollen, microspores and the like) seeds, plant cells and the progeny thereof.

By “plant CS” is meant a CS polypeptide that is naturally occurring in at least one plant species. The CS is from any plant, including monocots, dicots, C3 plants, C4 plants and/or plants that are classified as neither C3 nor C4 plants.

By “polypeptide” is meant a chain of at least four amino acids joined by peptide bonds. The chain is linear, branched, circular or combinations thereof. The polypeptides may contain amino acid analogs and other modifications, including, but not limited to glycosylated or phosphorylated residues.

As used herein, the term “SDS-PAGE” means sodium dodecyl sulfate-polyacrylimide gel electrophoresis.

The term “specific binding” refers to an interaction between CS and a molecule or compound, wherein the interaction is dependent upon the primary amino acid sequence or the conformation of CS.

The present invention discloses that antisense suppression of a chlorophyll synthase (CS) gene results in plants exhibiting one or more of chlorosis, reduced growth, and altered development. Thus, the present invention provides proteins encoded by CS genes as essential for normal plant growth and development, and as useful targets for the identification of herbicides. Accordingly, the present invention provides methods for identifying compounds that inhibit CS protein activity as candidate herbicides. CS enzyme activity comprises esterification of propionate moiety at position 17 of the chlorin ring in chlorophyllide (chelide) with a phospholipid substrate such as geranylgeranyl diphosphate or phytyl diphosphate with the requirement of an R conformation at position 13² of the isocyclic ring of the chlorophyllide. Identification of compounds that inhibit CS enzyme activity in the methods of the invention involves selectively measuring fluorescence due to product chlorophyll over that of substrate chelide. Compounds identified by the methods of the invention as being inhibitors of CS protein activity are useful as antibiotics and herbicides, especially as herbicides.

Addition of a solution comprising a water immiscible organic solvent and a water-soluble dye to the CS enzyme reaction incubations is used to advantage in the methods of the present invention to selectively suppress fluorescence of the CS chelide substrate over the CS chlorophyll product, enabling an efficient screening assay to identify CS inhibitory compounds. Thus, the methods of the present invention enable the concurrent testing of multiple compounds using a high throughput format such as with 96- or 384-well plates. In one embodiment, the invention provides a method for identifying an inhibitor of a CS enzyme, comprising: incubating a CS polypeptide with a chlorphyllide and a phospholipid substrate in the presence and absence of a test compound under conditions suitable for CS activity; adding to the incubation reactions a solution comprising a water immiscible organic solvent, a water-soluble alcohol, and a water-soluble dye that absorbs in the range of one or both of the excitation and emission wavelengths of the chlorphyllide substrate; and measuring the fluorescence of the incubation reactions at from about 650 to 750 nm, using from about 425 to 445 nm as excitation wavelength, wherein a decrease in the fluorescence in the presence of the test compound indicates that the compound is a CS inhibitor.

Selective suppression of the chlorophyllide substrate versus the chlorophyll product fluorescence, in the methods of the invention, is achieved by partitioning the chlorophyllide substrate and the dye into an aqueous phase and the chlorophyll product into a separate organic phase, such that only the substrate is subjected to the interfering properties of the dye. In this manner, the invention provides an efficient method for identifying CS inhibitory compounds that does not require manipulation of the incubation reactions to physically separate the product-containing organic phase from the substrate-containing aqueous phase. Thus, the methods of the present invention enable the concurrent testing of multiple compounds using a high throughput format such as with 96- or 384-well plates.

Inclusion of the water-soluble alcohol along with the water immiscible organic solvent in the methods of the invention aids the solubility of the chlorophyll product, allowing for efficient extraction of the chlorophyll product into the organic phase. Water-soluble alcohols useful in the methods of the invention include, but are not limited to, compounds such as methanol, ethanol, propanol, and isopropanol. In general, efficient extraction of the chlorophyll product into the organic phase is enabled by use of a 50% (V/V) or greater concentration of water-soluble alcohol.

Use of the phrase “a solution comprising a water immiscible organic solvent, a water-soluble alcohol, and a water-soluble dye” is not meant to be limiting, as the methods of the invention are equally amenable to addition of each of the three reagents in single solutions or in combinations of one or more solutions. In addition, the methods of the invention are presented above such that the water-soluble dye is added subsequent to the enzyme incubation, although subsequent addition of the dye in the manner described above is not a requirement. In an alternative embodiment the water-soluble dye may be present throughout the enzyme reaction incubation, as the dye does not interfere in the enzyme reaction and is only necessary for measurement of product fluorescence. Accordingly, the methods of the invention include methods for identifying an inhibitor of a CS enzyme, comprising: incubating a CS polypeptide with a chlorphyllide and a phospholipid substrate in the presence of a water-soluble dye that absorbs in the range of one or both of the excitation and emission wavelength ranges of the chlorphyllide substrate, and in the presence and absence of a test compound under conditions suitable for CS activity; adding to the incubation reactions a solution comprising a water immiscible organic solvent and a water-soluble alcohol; and measuring the fluorescence of the incubation reactions at from about 650 to 750 nm, using from about 425 to 445 nm as excitation wavelength, wherein a decrease in the fluorescence in the presence of the test compound indicates that the compound is a CS inhibitor.

Organic solvents useful in the methods of the invention are those solvents in which the CS chlorophyll product is soluble and the chelide substrate is not. Water immiscible organic solvents useful in the methods of the invention are those capable of selectively extracting chlorophyll in the presence of chlorophyllide from an aqueous solution containing a water-soluble alcohol of the invention. The water immiscible organic solvents of the invention have a relatively low vapor pressure and high boiling point, such that the solvents are compatible with a high throughput format. The organic solvents of the invention include, but are not limited to, hydrocarbons such as hexane, heptane, octane, decane, dodecane, tridecane and similar hydrocarbons of higher carbon content. Similar hydrocarbons that are unsaturated liquid hydrocarbons are also useful in the methods of the invention, as are solutions of liquid hydrocarbons into which a small amount of a solid hydrocarbon (i.e. a wax) has been added.

Water-soluble dyes useful in the methods of the invention are those dyes that have solubility properties similar to the chelide substrate. For example, dyes useful in the methods of the invention will partition with the chelide substrate into aqueous phase while the chlorophyll product partitions into organic phase. In addition to having specific solubility properties, dyes useful in the methods of the invention also posses spectral properties such that the dyes absorb in the range of one or both of the excitation and emission wavelength ranges of the substrate chelide. Examples of dyes useful in the methods of the invention, having suitable solubility and spectral characteristics, are Malachite Green and Palatine Fast Yellow BLN.

In one embodiment of the invention, the organic solvent is dodecane. In another embodiment of the invention, the water-soluble dye is Malachite Green. In another embodiment of the invention, the phospholipid substrate is geranylgeranyl (GGPP). In another embodiment of the invention, the phospholipid substrate is phytyl diphosphate. In another embodiment of the invention, the incubation of the CS polypeptide with the substrates in the presence and absence of the test compound is carried out in a solution comprising ethanol, such that the increased solubility of the chelide substrate in water due to the presence of the ethanol results in enhancement of the CS polypeptide activity. In another embodiment of the invention, the fluorescence of the incubation reactions is measured at about 665 nm using about 440 nm as excitation wavelength. In another embodiment of the invention, the fluorescence of the incubation reactions is measured at about 665 nm using about 430 nm as excitation wavelength. In another embodiment of the invention, measurement of the fluorescence of the incubation reactions is performed about three hours after addition of the solution comprising the organic solvent and the water-soluble dye.

In the methods of the invention, incubation of the CS polypeptide with the substrates in the presence and absence of the test compound is performed under conditions suitable for the CS enzyme activity. A wide range of conditions are suitable for promoting CS enzyme activity, including a range of incubation times and temperatures, buffers, and substrate concentrations. Choice of one particular parameter may influence the choice of another parameter such as use of a lower incubation temperature resulting in a longer incubation time. Therefore, the methods of the invention include a wide range of CS enzyme activity conditions. In one embodiment of the invention, the incubation of the CS polypeptide with the substrates in the presence and absence of the test compound is performed under conditions suitable for the CS enzyme activity at a pH range from about 7.5-8.5. In another embodiment of the invention, the incubation of the CS polypeptide with the substrates in the presence and absence of the test compound is performed under conditions suitable for the CS enzyme activity at a pH of about 8.0. In another embodiment of the invention, the CS polypeptide is incubated with substrate chlorophyllide within a range of 1.0-5.0 μM and substrate geranylgeranyl diphosphate within a range of 5.0-50 μM in 50 mM Tris buffer at pH 8.0 containing about 1 mM Mg⁺², about 5% (V/V) ethanol, and about 0.01% Tween 20.

The methods of the invention are amenable to high throughput formats. Thus, another embodiment of the invention is a method for the concurrent testing of a plurality of compounds for an ability to inhibit CS enzyme activity, comprising: incubating a plurality of test compounds in a multi-well format, individually or in mixtures, with a CS polypeptide and a chlorophyllide and phopholipid substrate under conditions suitable for CS activity; incubating in at least one of the wells the CS polypeptide and substrates under conditions suitable for CS activity in the absence of a test compound; adding to the incubation reactions of each of the wells a solution comprising a water immiscible organic solvent, a water-soluble alcohol and a water-soluble dye that absorbs in the range of one or both of the excitation and emission wavelength ranges of the chlorphyllide substrate; measuring the fluorescence of the wells at about 665 nm using about 440 nm as excitation wavelength; and comparing the fluorescence of the wells comprising the CS in the presence and in the absence of the test compound(s), wherein a relative decrease in fluorescence for the wells comprising the test compound(s), indicates that at least one of the test compounds comprised within is a CS inhibitor.

In another embodiment of the invention, selective measurement of the fluorescence of the product chlorophyll over the substrate chlorophyllide is achieved without the use of the water-soluble dye. In this embodiment of the invention, the fluorescence emission wavelength of the substrate chlorophyllide is shifted towards the blue end of the spectrum by the presence of the immiscible organic solvent, whereas the emission of the product chlorophyll is unaffected by the presence of the organic solvent. In this manner, selective measurement of the fluorescence of the product chlorophyll is possible in the presence of the substrate chlorophyllide.

In one embodiment of the invention, the CS has the amino acid sequence of a naturally occurring CS found in a plant or microorganism. In another embodiment of the invention, the CS has an amino acid sequence derived from a naturally occurring sequence. In another embodiment, the CS is a plant CS. In another embodiment, the plant CS is a from a dicot plant. In another embodiment, the plant CS is a from a monocot plant. In another embodiment, the CS is an Arabidopsis CS which includes, but is not limited to, Arabidopsis arenosa, Arabidopsis bursifolia, Arabidopsis cebennensis, Arabidopsis croatica, Arabidopsis griffithiana, Arabidopsis halleri, Arabidopsis himalaica, Arabidopsis korshinskyi, Arabidopsis lyrata, Arabidopsis neglecta, Arabidopsis pumila, Arabidopsis suecica, Arabidopsis thaliana and Arabidopsis wallichii. In another embodiment, the CS is a fungal CS.

Examples of particular CS polypeptides of the invention include, but are not limited to Arabidopsis thalianana CS (SEQ ID NO:1; Accession No. NP_{—}190750); Oryza sativa CS (SEQ ID NO:2; Accession No. AAL79703); Avena sativa CS (SEQ ID NO:3; Accession No. CAB85464); and Arabidopsis thaliana CS fusion protein (SEQ ID NO:4).

In various embodiments, the CS can be from barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium, and the like.

Polypeptides consisting essentially of SEQ ID NO:1-3 are also useful in the methods of the invention. For the purposes of the present invention, a polypeptide consisting essentially of any one of SEQ ID NOS:1-3 has at least 90% sequence identity with the particular SEQ ID NO:1-3 and at least 10% of the activity of the particular SEQ ID NO:1-3. For example, a polypeptide consisting essentially of SEQ ID NO:1 has at least 90% sequence identity with Arabidopsis thaliana CS (SEQ ID NO:1) and at least 10% of the activity of SEQ ID NO:1. A polypeptide consisting essentially of SEQ ID NO:1 has at least 90%, 91%, 92%, 93%, 94%, 95%, 96%, 97%, 98%, or 99% sequence identity with SEQ ID NO:1 and at least 25%, 50%, 75%, or 90% of the activity of Arabidopsis thaliana CS (SEQ ID NO:1). Examples of polypeptides consisting essentially of any one of SEQ ID NOS:1-3 include, but are not limited to, polypeptides having the amino acid sequence of any one of SEQ ID NOS:1-3 with the exception that one or more of the amino acids of any particular SEQ ID NO are substituted with structurally similar amino acids providing a “conservative amino acid substitution.” Conservative amino acid substitutions are well known to those of skill in the art. Particular examples of polypeptides consisting essentially of SEQ ID NO:1 include polypeptides having 1, 2, or 3 conservative amino acid substitutions relative to SEQ ID NO:1.

Other examples of polypeptides consisting essentially of any one of SEQ ID NOS:1-3 include polypeptides having the sequence of any one of SEQ ID NOS:1-3, but with truncations at either or both the 3′ and the 5′ end of the particular SEQ ID NO. For example, polypeptides consisting essentially of SEQ ID NO:1 include polypeptides having 1, 2, or 3 amino acids residues removed from either or both 3′ and 5′ ends relative to SEQ ID NO:1. In addition, CS polypeptides consisting essentially of SEQ ID NO:1 can be fusion proteins, such as SEQ ID NO:4, in which a CS polypeptide is fused with another polypeptide or amino acid sequence to aid in secretion and/or purification, as is known to those of skill in the art. SEQ ID NO:4 is an amino-terminal CS fusion polypeptide (6-His tag, thrombin cleavage site, S-tag, and enterokinase fused to Arabidopsis thaliana CS, in that order, where the first 57 amino acids of the Arabidopsis thaliana CS protein have been deleted).

Test compounds that are identified by the methods of the present invention to be inhibitors of CS activity are further tested as herbicides by direct application to a plant or plant cell, or expression therein, and monitoring the plant or plant cell for changes or decreases in growth, development, viability or alterations in gene expression. A decrease in growth occurs where the herbicide candidate causes at least a 10% decrease in the growth of the plant or plant cells, as compared to the growth of the plants or plant cells in the absence of the herbicide candidate. A decrease in viability occurs where at least 20% of the plants cells, or portions of the plant contacted with the herbicide candidate, are nonviable. Preferably, the growth or viability will be decreased by at least 40%. More preferably, the growth or viability will be decreased by at least 50%, 75%, or at least 90% or more. Methods for measuring plant growth and cell viability are known to those skilled in the art. It is possible that a test compound may have herbicidal activity only for certain plants or certain plant species.

For use in the screening assays of the invention, CS protein and derivatives thereof may be isolated from a plant or may be recombinantly produced in and isolated from a plant, bacteria or eukaryotic cell culture. Preferably CS proteins are produced using a baculovirus, E. coli or yeast expression system. Methods for generating isolated CS polypeptide are found, for example, in Schmid et al. (2001), supra, and Oster & Rudiger (1997), supra, and herein at Examples 1 and 2. Other methods for the purification of CS proteins and polypeptides are known to those skilled in the art.

Chemicals, compounds, or compositions identified by the above methods as modulators of CS activity are useful for controlling plant growth. For example, compounds that inhibit plant growth are applied to a plant to prevent plant growth. Thus, the invention provides a method for inhibiting plant growth, comprising contacting a plant with a compound identified by the methods of the invention as having herbicidal activity.

Test compounds identified by the methods of the invention as herbicide candidates are useful for controlling the growth of undesired plants, including monocots, dicots, C3 plants, C4 plants, and plants that are neither C3 nor C4 plants. Examples of undesired plants include, but are not limited, to barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium, and the like.

### EXPERIMENTAL

### Example 1

### Construction of a Transgenic Plant Expressing a “Driver”

The “Driver” is an artificial transcription factor comprising a chimera of the DNA-binding domain of the yeast GAL4 protein (amino acid residues 1-147) fused to two tandem activation domains of herpes simplex virus protein VP16 (amino acid residues 413-490). Schwechheimer et al. (1998) 36 Plant Mol. Biol. 195-204. This chimeric driver is a transcriptional activator specific for promoters having GAL4 binding sites. Expression of the driver is controlled by two tandem copies of the constitutive CaMV 35S promoter.

The driver expression cassette was introduced into Arabidopsis thaliana by agroinfection. Transgenic plants that stably expressed the driver transcription factor were obtained.

### Example 2

### Construction of a CS Antisense Expression Cassette in a Binary Vector

Fragments of CS Arabidopsis thaliana cDNA corresponding to SEQ ID NO:1 were ligated into the multiple cloning site of an E.coli/Agrobacterium binary vector in the antisense orientation to yield an antisense expression cassette and a constitutive chemical resistance expression cassette located between right and left T-DNA borders. For analysis by transactivation, transcription of the antisense RNA was placed under control of an artificial promoter active only in the presence of the driver transcription factor described above. The artificial promoter contains four contiguous binding sites for the GAL4 transcriptional activator upstream of a minimal promoter comprising a TATA box.

### Example 3

### Transformation of Agrobacterium with the CS Antisense Expression Cassette

The binary vector described in Example 2 containing the CS expression cassette was separately transformed into Agrobacterium tumefaciens by electroporation. Transformed Agrobacterium colonies were isolated using chemical selection. DNA was prepared from purified resistant colonies and the inserts were amplified by PCR and sequenced to confirm sequence and orientation.

### Example 4

### Generation of Transgenic Arabidopsis Plants Containing a CS Antisense Expression Construct

The antisense expression cassette corresponding to the CS Arabidopsis gene sequence was introduced into Arabidopsis transgenic driver line plants (described above) by the following method. Five days prior to agroinfection, the primary inflorescence of Arabidopsis thaliana plants grown in 2.5 inch pots were clipped to enhance the emergence of secondary bolts.

At two days prior to agroinfection, 5 ml LB broth (10 g/L Peptone, 5 g/L Yeast extract, 5 g/L NaCl, pH 7.0 plus 25 mg/L kanamycin added prior to use) was inoculated with a clonal glycerol stock of Agrobacterium carrying the desired DNA. The cultures were incubated overnight at 28° C. at 250 rpm until the cells reached stationary phase. The following morning, 200 ml LB in a 500 ml flask was inoculated with 500 μl of the overnight culture and the cells were grown to stationary phase by overnight incubation at 28° C. at 250 rpm. The cells were pelleted by centrifugation at 8000 rpm for 5 min. The supernatant was removed and excess media was removed by setting the centrifuge bottles upside down on a paper towel for several minutes. The cells were then resuspended in 500 ml infiltration medium (autoclaved 5% sucrose) and 250 μl/L SILWET L-77 (84% polyalkyleneoxide modified heptamethyltrisiloxane and 16% allyloxypolyethyleneglycol methyl ether), and transferred to a one-liter beaker.

The previously clipped Arabidopsis plants were dipped into the Agrobacterium suspension so that all above ground parts were immersed and agitated gently for 10 sec. The dipped plants were then covered with a tall clear plastic dome to maintain the humidity, and returned to the growth room. The following day, the dome was removed and the plants were grown under normal light conditions until mature seeds were produced. Mature seeds were collected and stored desiccated at 4° C.

Transgenic Arabidopsis T1 seedlings were selected for the CS construct. Approximately 70 mg seeds from an agrotransformed plant were mixed approximately 4:1 with sand and placed in a 2 ml screw cap cryo vial. One vial of seeds was then sown in a cell of an 8 cell flat. The flat was covered with a dome, stored at 4° C. for 3 days, and then transferred to a growth room. The domes were removed when the seedlings first emerged. After the emergence of the first primary leaves, the flat was sprayed uniformly with a herbicide corresponding to the chemical resistance marker plus 0.005% Silwet (50 μl/L) until the leaves were completely wetted. The spraying was repeated for the following two days.

Ten days after the first spraying resistant plants were transplanted to 2.5 in round pots containing moistened sterile potting soil. The transplants were then sprayed with herbicide and returned to the growth room. The herbicide resistant plants represented stably transformed T1 plants.

### Example 5

### Effect of CS Antisense Expression by Transactivation in Arabidopsis Seedlings

The T1 antisense target plants from the transformed plant line obtained in Example 4 were grown to maturity. T2 seed was harvested from these plants and subjected to a plate assay to observe seedling growth over a 2-week period. Seedlings were inspected for growth and development. One T2 line expressing an antisense transcript corresponding to chlorophyll synthase TIGR ID: At3g51820 exhibited chlorosis and whole seedling reduced size, indicating that the gene is essential for normal plant growth and development.

### Example 6

### Cloning of a cDNA Encoding CS Protein

Total RNA was collected from 14-day-old Arabidopsis thaliana seedlings using published protocols, and reagents (Trizol) from Life Technologies, Inc. (Rockville, Md.). 1 μg of total RNA was incubated with 10 pmol of custom oligo: TCAGTGTTGCGATGCTAATGCCGT (SEQ ID NO:5) in a reverse transcription reaction (Thermoscript RT kit, Life Technologies) according to the manufacture's recommendations. Polymerase chain reaction (PCR) was carried out in a total volume of 50 μl with the following reagents: 2 μl of above RT reaction mixture, 20 mM Tris-HCl, pH 8.8, 2 mM MgSO₄, 10 mM KCl, 10 mM (NH₄)₂SO₄, 0.1% Triton X-100, 0.1 mg/ml BSA, 10 mM dNTPs, 15 pmol of each primer:

| | |
|---|---|
| CCGGGATCCGCGGCGGAGACTGATACTGATAAA | (SEQ ID NO:6) |
| and | |
| CCGCTCGAGTCAGTGTTGCGATGCTAATGCCGT | (SEQ ID NO:7) |

and 2.5 units pfu Turbo polymerase (Stratagene, USA). PCR cycling was as follows: 94° C. (3 min), 55° C. (1 min), 68° C. (3 min) for 1 cycle, 94° C. (45 sec), 55° C. (30 sec), 68° C. (2 min) for 30 cycles, 68° C. (10 min). The resulting PCR product and plasmid pET30a (+) (Novagen, Madison, Wis.) were digested with restriction endonucleases BamHI and XhoI, as directed by the manufacturer (Life Technologies). Ligation of the two linear DNA's into the resulting recombinant clone pET30a-tCS was accomplished by following instructions included with T4 DNA ligase (New England Biolabs). The integrity of the above clone was verified by DNA sequence analysis to confirm the sequence set forth in SEQ ID NO:4.

### Example 7

### Expression and Isolation of Recombinant CS Protein

Clone pET30a-tCS was transformed into E. coli Rosetta (DE3) pLysS (Novagen Cat. No. 70956-4) following manufacturer's instructions. Transformed bacteria were grown in LB liquid media (10 grams each tryptone and NaCl; 5 grams yeast extract; H₂O to one liter) supplemented with 34 micrograms/milliliter chloramphenicol and 50 micrograms/milliliter kanamycin, at 37° C. to an optical density of 0.6 at 600 nanometers. At this point, isopropylthio-Beta-galactoside (IPTG) was added to a final concentration of 1 mM and the culture was incubated at 23° C. for 16 additional hours. Bacteria were pelleted via centrifugation, the supernatant discarded, and the pellet frozen to −80° C.

The pellet from 1L culture (induced overnight at room temperature with 1 mM IPTG) was re-suspended in 20 ml of 50 mM HEPES pH 8.0 containing complete EDTA free protease inhibitor (Rosch Cat No. 1873580; 1 tablet per 50 ml), Benzonase (Novagen Cat No. 70746; 1 μl/ml), Lysozyme (Calbiochem; 1 mg/ml). Lysis was performed by sonicating the cells on ice for 5 minutes followed by two freeze thaw cycles (Schmid et al. (2001), supra). The lysate was cleared by centrifugation at 2400×g for 20 minutes. Supernatant obtained from this step was centrifuged at 15000×g for 30 minutes to pellet the chlorophyll synthase-containing membrane. The pellet thus obtained was then suspended in 3 ml of 50 mM HEPES pH 8.0 and centrifuged as before. Finally the membrane fraction was suspended in 3 ml of 50 mM HEPES pH 8.0 and the isolation of CS fusion protein (SEQ ID NO:4) was affirmed by PAGE and western blot analysis (data not shown). The isolated CS fusion protein was stored at −80° C., and exhibited excellent retention of enzymatic activity following multiple freeze-thaw cycles (data not shown).

### Example 8

### Assay for CS Enzyme Activity and Inhibitors Thereof

An assay suitable for high throughput screening of compounds for the identification of inhibitors of CS activity was performed as follows. 20 μl of substrate solution (5 μM chelide, 20 μM GGPP in assay buffer, 50 mM Tris pH 8 containing 1 mM MgSO₄, 5% (v/v) ethanol, and 0.01% (v/v) Tween 20) was mixed with 1 μg of CS enzyme (Example 7) in an equal volume of assay buffer (or a no enzyme control), and the reaction was incubated for 30 min at room temperature. The reaction was stopped by the addition of 60 μl of 5 mM malachite green in 50% (v/v) dodecane-ethanol. The plate was allowed to sit for 4 hr at room temperature, after which fluorescence was measured using 440 nm excitation and 665 nm emission). FIG. 3 shows the results of the above-described assay in which the enzyme was added in assay buffer containing varying amounts of DMSO, to test the effect of DMSO on enzyme activity. The upper and the lower curves in FIG. 3 depict the reaction in the presence and absence of enzyme, respectively.

1. A method for identifying inhibitors of a chlorophyll synthase (CS) enzyme, comprising:
a) incubating a CS polypeptide with a chlorphyllide and a phospholipid substrate in the presence and absence of a test compound under conditions suitable for CS activity;
b) adding to the incubation reactions a solution comprising a water immiscible organic solvent, a water-soluble alcohol, and a water-soluble dye that absorbs in the range of one or both of the excitation and emission wavelengths of the chlorphyllide substrate; and
c) measuring the fluorescence of the incubation reactions at from about 650 to 750 nm, using from about 425 to 445 nm as excitation wavelength, wherein a decrease in the fluorescence in the presence of the test compound indicates that the compound is a CS inhibitor.

2. The method of claim 1, wherein the CS is a plant CS.

3. The method of claim 2, wherein the plant is a dicot.

4. The method of claim 2, wherein the plant is a monocot.

5. The method of claim 2, wherein the CS is an Arabidopsis CS.

6. The method of claim 2, wherein the CS is SEQ ID NO:1.

7. The method of claim 2, wherein the CS is a CS polypeptide consisting essentially of SEQ ID NO:1.

8. The method of claim 1, wherein the CS is a fungal CS.

9. The method of claim 1 wherein the fluorescence is measured at about 665 nm using about 440 nm as excitation wavelength.

10. The method of claim 1 wherein the water immiscible organic solvent is dodecane, the water-soluble dye is Malachite Green, the water-soluble alcohol is ethanol and the phospholipid substrate is geranylgeranyl diphosphate.

11. A method for identifying inhibitors of a chlorophyll synthase (CS) enzyme, comprising:
a) incubating a CS polypeptide with a chlorphyllide and a phospholipid substrate in the presence of a water-soluble dye that absorbs in the range of one or both of the excitation and emission wavelength ranges of the chlorphyllide substrate, and in the presence and absence of a test compound under conditions suitable for CS activity;
b) adding to the incubation reactions a solution comprising a water immiscible organic solvent and a water-soluble alcohol; and
c) measuring the fluorescence of the incubation reactions at from about 650 to 750 nm, using from about 425 to 445 nm as excitation wavelength, wherein a decrease in the fluorescence in the presence of the test compound indicates that the compound is a CS inhibitor.

12. The method of claim 11, wherein the CS is a plant CS.

13. The method of claim 12, wherein the plant is a dicot.

14. The method of claim 12, wherein the plant is a monocot.

15. The method of claim 12, wherein the CS is an Arabidopsis CS.

16. The method of claim 12, wherein the CS is SEQ ID NO:1.

17. The method of claim 12, wherein the CS is a CS polypeptide consisting essentially of SEQ ID NO:1.

18. The method of claim 11, wherein the CS is a fungal CS.

19. The method of claim 11 wherein the fluorescence is measured at about 665 nm using about 440 nm as excitation wavelength.

20. The method of claim 11 wherein the water immiscible organic solvent is dodecane, the water-soluble dye is Malachite Green, the water-soluble alcohol is ethanol and the phospholipid substrate is geranylgeranyl diphosphate.

21. A method for concurrently testing a plurality of compounds as inhibitors of a chlorophyll synthase (CS) enzyme, comprising:
a) incubating a plurality of test compounds in a multi-well format, individually or in mixtures, with a CS polypeptide and a chlorophyllide and phopholipid substrate under conditions suitable for CS activity;
b) incubating in at least one of the wells the CS polypeptide and the substrates under conditions suitable for CS activity in the absence of a test compound;
c) adding to the incubation reactions of each of the wells a solution comprising a water immiscible organic solvent, a water-soluble alcohol and a water-soluble dye that absorbs in the range of one or both of the excitation and emission wavelength ranges of the chlorphyllide substrate;
d) measuring the fluorescence of the wells from about 650 to 750 nm using from about 425 to 445 nm as excitation wavelength; and
e) comparing the fluorescence of the wells comprising the CS in the presence and in the absence of the test compound(s), wherein a relative decrease in fluorescence for the wells comprising the test compound(s), indicates that at least one of the test compounds comprised within is a CS inhibitor.

22. The method of claim 21, wherein the CS is a plant CS.

23. The method of claim 22, wherein the plant is a dicot.

24. The method of claim 22, wherein the plant is a monocot.

25. The method of claim 22, wherein the CS is an Arabidopsis CS.

26. The method of claim 22, wherein the CS is SEQ ID NO:1.

27. The method of claim 22, wherein the CS is a CS polypeptide consisting essentially of SEQ ID NO:1.

28. The method of claim 21, wherein the CS is a fungal CS.

29. The method of claim 21 wherein the fluorescence is measured at about 665 nm using about 440 nm as excitation wavelength.

30. The method of claim 21 wherein the water immiscible organic solvent is dodecane, the water-soluble dye is Malachite Green, the water-soluble alcohol is ethanol and the phospholipid substrate is geranylgeranyl diphosphate.

31. A method for concurrently testing a plurality of compounds as inhibitors of a chlorophyll synthase (CS) enzyme, comprising:
a) incubating a plurality of test compounds in a multi-well format, individually or in mixtures, with a CS polypeptide and a chlorophyllide and phopholipid substrate under conditions suitable for CS activity, and with a water-soluble dye that absorbs in the range of one or both of the excitation and emission wavelength ranges of the chlorphyllide substrate;
b) incubating in at least one of the wells the CS polypeptide, the substrates and the water-soluble dye under conditions suitable for CS activity in the absence of a test compound;
c) adding to the incubation reactions of each of the wells a solution comprising a water immiscible organic solvent and a water-soluble alcohol;
d) measuring the fluorescence of the wells from about 650 to 750 nm using from about 425 to 445 nm as excitation wavelength; and
e) comparing the fluorescence of the wells comprising the CS in the presence and in the absence of the test compound(s), wherein a relative decrease in fluorescence for the wells comprising the test compound(s), indicates that at least one of the test compounds comprised within is a CS inhibitor.

32. The method of claim 31, wherein the CS is a plant CS.

33. The method of claim 32, wherein the plant is a dicot.

34. The method of claim 32, wherein the plant is a monocot.

35. The method of claim 32, wherein the CS is an Arabidopsis CS.

36. The method of claim 32, wherein the CS is SEQ ID NO:1.

37. The method of claim 32, wherein the CS is a CS polypeptide consisting essentially of SEQ ID NO:1.

38. The method of claim 31, wherein the CS is a fungal CS.

39. The method of claim 31 wherein the fluorescence is measured at about 665 nm using about 440 nm as excitation wavelength.

40. The method of claim 31 wherein the water immiscible organic solvent is dodecane, the water-soluble dye is Malachite Green, the water-soluble alcohol is ethanol and the phospholipid substrate is geranylgeranyl diphosphate.


SEQUENCE LISTING (7 sequences)

SEQ ID NO: 1 - protein, 387 aa, Arabidopsis thaliana
MTSILNTVSTIHSSRVTSVDRVGVLSLRNSDSVEFTRRRSGFSTLIYESPGRRFVVRAAETDTDKVKSQTPDKAPAGGSSINQLLGIKGASQETNKWKIRLQLTKPVTWPPLVWGVVCGAAASGNFHWTPEDVAKSILCMMMSGPCLTGYTQTINDWYDRDIDAINEPYRPIPSGAISEPEVITQVWVLLLGGLGIAGILDVWAGHTTPTVFYLALGGSLLSYIYSAPPLKLKQNGWVGNFALGASYISLPWWAGQALFGTLTPDVVVLTLLYSIAGLGIAIVNDFKSVEGDRALGLQSLPVAFGTETAKWICVGAIDITQLSVAGYLLASGKPYYALALVALIIPQIVFQFKYFLKDPVKYDVKYQASAQPFLVLGIFVTALASQH

SEQ ID NO: 2 - protein, 541 aa, Orzya sativa
MTTVASLSLLPHLLIKPSFRCCSRKGVGRYGGIKVYAVLGDDGADYAKNNAWEALFHVDDPGPRVPIAKGKFLDVNQALEVVRFDIQYCDWRARQDLLTIMVLHNKVVEVLNPLAREFKSIGTLRKELAELQEELAKAHNQVHLSETRVSSALDKLAQMETLVNDRLLQDGGSSASTAECTSLAPSTSSASRVVNKKPPRRSLNVSGPVQPYNPSLKNFWYPVAFSSDLKDDTMVPIDCFEEQWVIFRGKDGRPGCVMNTCAHRACPLHLGSVNEGRIQCPYHGWEYSTDGKCEKMPSTKMLNVRIRSLPCFEQEGMVWIWPGNDPPKSTIPSLLPPSGFTIHAEIVMELPVEHGLLLDNLLDLAHAPFTHTSTFAKGWSVPSLVKFLTPSSGLQGYWDPYPIDMEFRPPCMVLSTIGISKPGKLEGKSTKQCSTHLHQLHICLPSSRNKTRLLYRMSLDFAPWIKHVPFMHILWSHFAEKVLNEDLRLVLGQQERMINGANVWNWPVSYDKLGIRYRLWRDAIERGVDRLPFSNQSESGS

SEQ ID NO: 3 - protein, 378 aa, Avena sativa
MATSHPLAAAAATSSSSATFRPPLRFLSSPPSSLTLNRRRSFPVVCAADADAKETTKKPTIPDKAPAAGSSFNQLLGIKGAKQETNIWKIRLQLTKPVTWPPLVWGVLCGAAASGNFHWTVEDVTKSIVCMLMSGPCLTGYTQTINDWYDRDIDAINEPYRPIPSGAISENEVITQIWVLLLGGLGLGALLDIWAGHDFPIIFYLALGGSLLSYIYSAPPLKLKQNGWIGNFALGASYIGLPWWAGQALFGTLTPDIVVLTCLYSIAGLGIAIVNDFKSIEGDRTLGLQSLPVAFGMETAKWICVGAIDITQLSVAAYLLSTGKLYYALALLGLTIPQVILQFQYFLKDPVKYDVKYQASAQPFFVFGLLVTALATSH

SEQ ID NO: 4 - protein, 380 aa, Artificial
  Arabidopsis thaliana fusion protein
MHHHHHHSSGLVPRGSGMKETAAAKFERQHMDSPDLGTDDDDKAMADIGSAAETDTDKVKSQTPDKAPAGGSSINQLLGIKGASQETNKWKIRLQLTKPVTWPPLVWGVVCGAAASGNFHWTPEDVAKSILCMMMSGPCLTGYTQTINDWYDRDIDAINEPYRPIPSGAISEPEVITQVWVLLLGGLGIAGILDVWAGHTTPTVFYLALGGSLLSYIYSAPPLKLKQNGWVGNFALGASYISLPWWAGQALFGTLTPDVVVLTLLYSIAGLGIAIVNDFKSVEGDRALGLQSLPVAFGTETAKWICVGAIDITQLSVAGYLLASGKPYYALALVALIIPQIVFQFKYFLKDPVKYDVKYQASAQPFLVLGIFVTALASQH

SEQ ID NO: 5 - DNA, 24 bp, Artificial
  RT-PCR primer
tcagtgttgcgatgctaatgccgt

SEQ ID NO: 6 - DNA, 33 bp, Artificial
  PCR primer 1
ccgggatccgcggcggagactgatactgataaa

SEQ ID NO: 7 - DNA, 33 bp, Artificial
  PCR primer 2
ccgctcgagtcagtgttgcgatgctaatgccgt